(19) United States
(12) Patent Application Publication
(10) Pub. No.: US 2025/0241830 A1
(43) Pub. Date: Jul. 31, 2025

(54) MOLYBDENUM AND HYDROXYAPATITE COMPOSITIONS AND METHODS

(71) Applicant: Jennifer Jablow, Watermill, NY (US)

(72) Inventor: Jennifer Jablow, Watermill, NY (US)

(21) Appl. No.: 19/037,173

(22) Filed: Jan. 25, 2025

Related U.S. Application Data
(60) Provisional application No. 63/624,868, filed on Jan. 25, 2024.

(51) Int. Cl. A61K 6/838; A61K 6/20; A61K 8/19; A61Q 11/00

(52) U.S. Cl. CPC A61K 6/838; A61K 6/20; A61K 8/19; A61Q 11/00

(57) ABSTRACT

The present disclosure relates to compositions and methods for strengthening teeth including molybdenum and a re-mineralization and anti-sensitivity agent.

### FIELD

The present disclosure includes compositions and methods for strengthening teeth.

### BACKGROUND

Fluoride has been used historically to strengthen a tooth's hard outer surface called enamel. Various sources of fluoride include drinking tap water in communities where the public water system adds fluoride and by using fluoride-containing toothpaste and mouth rinse, as well as from certain foods and beverages.

Molybdenum is a naturally occurring essential trace element with several biological functions and therapeutic uses and may have a cariostatic effect and could be used as an alternative to fluoride.

### SUMMARY

In one embodiment, a composition is provided. The composition includes molybdenum and a remineralization and anti-sensitivity agent.

In another embodiment, a method for strengthening teeth is provided. The method includes applying a composition to one or more dental surfaces in the mouth of a patient, the composition including molybdenum and a remineralization and anti-sensitivity agent.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Advantages and other aspects of the exemplary embodiments included herein will become apparent from the following detailed description. Additional aspects and advantages of the disclosure will be set forth in the description that follows, and in part will be evident from the description, or may be learned by practice of the disclosure. Moreover, the aspects and advantages of the disclosure may be realized and obtained by means of the instrumentalities and combinations particularly pointed out in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “%”, “% by weight”, “weight %” and “wt %” are all intended to mean unless otherwise stated, percents by weight based upon a total weight of 100% end composition weight. Thus 10% by weight means that the component constitutes 10 wt. parts out of every 100 wt. parts of total composition.

The term “dental surface” means soft or hard tissue of the oral environment including gums or a natural tooth surface (e.g., dentin or enamel) including the outer surface of a tooth (e.g., the hard outer surface of the tooth, i.e., the enamel also referred to as the enamel layer).

The term “dental tissue” means the hard and soft tissues of the mouth.

All of the embodiments included here are with the proviso that the sum of ingredients in the exemplary compositions does not exceed 100%.

Aspects of the present disclosure include embodiments including various compositions used for strengthening the hard outer surface of the teeth (e.g., the enamel barrier) and the benefit of preventing or lowering the risk of cavities as well as associated methods that can include making the embodiments of the present disclosure and methods of using same including a teeth outer surface strengthening agent.

The aspects of the present disclosure are directed to compositions as well as methods of making and using compositions of the present disclosure that have dental benefits including strengthening the hard outer surface of the teeth (e.g., the enamel layer) and the benefit of preventing or lowering the risk of cavities using a teeth strengthening agent and can include molybdenum and re-mineralization agent. Compositions of the present disclosure include compositions and components thereof that are applied to dental surfaces.

Embodiments of the present disclosure include molybdenum as a natural alternative to fluoride. Molybdenum may prevent or lower the risk of cavities by strengthening the hard outer surface of the teeth (e.g., the enamel layer), making it more resistant to acid attack and possibly prevent dental cavities. Molybdenum can be found commercially as sodium molybdate, ammonium molybdate, molybdenum glycinate, molybdenum citrate, and molybdenum chloride and can be used in embodiments of the present disclosure singularly or as chemically acceptable mixtures thereof. The amount of molybdenum can be in the range of from about 0.01 wt % to about 10 wt % or about 0.025 wt % to about 2.0 wt %.

Embodiments of the present disclosure include a re-mineralization agent that may prevent or lower the risk of cavities that may also be an anti-sensitivity agent. HAP (hydroxyapatite) is a dental agent capable of re-mineralizing enamel and is an anti-sensitivity agent. The HAP can be nano, micro or regular size or a mixture of at least two of the sizes and can be in any shape (e.g., a sphere or rod shape) or a combination of shapes. The amount of HAP can be in the range of from about 0.1 wt % to about 20 wt % or about 1 wt % to about 10 wt %. In certain embodiments of the present disclosure, such a re-mineralizing agent can also include a phosphate compound, a calcium compound, a calcium phosphate compound, and a caseinate. The phosphate compound can include, for example, a monobasic phosphate compound, a dibasic phosphate compound, a tribasic phosphate compound, calcium glycerophosphate, calcium acetate and combinations thereof.

Embodiments of the present disclosure can also include additional ingredients such as, for example, at least one flavoring agent and/or at least one sweetening agent.

Embodiments of the present disclosure may include one or more flavoring agents. Flavoring agents can include, for example, xylitol or stevia, a mint oil (such as, for example, spearmint (e.g. spearmint native oil) or peppermint (e.g., peppermint oil)) and mixtures thereof. Flavoring agents can be in the range of from about 0.1 wt % to about 5 wt %, by weight of the composition.

Embodiments of the present disclosure may include one or more sweetening agents. Sweetening agents can include, for example, saccharin, dextrose, sucrose, sucralose, lactose, maltose, levulose, aspartame, stevia, erythritol, xylitol, sodium cyclamate, D-tryptophan, dihydrochalcones, acesulfame, glycine, monk fruit and mixtures thereof, in particular, stevia, xylitol and mixtures thereof. Sweetening agents can be generally used at levels of from about 0.005 wt % to about 10 wt %, by weight of the composition, from 0.1 wt % to about 2 wt % or from about 2 wt % to about 3 wt %. Xylitol may also fight acid deterioration of the outer surface of the teeth and aid in the remineralizing process.

Embodiments of the present disclosure can also include ozone in the form that can be in an ozonated oil (also referred to as ozonide oil) such as olive oil, castor oil, coconut, hemp oil, sesame oil, sunflower oil, canola oil, avocado oil, grapeseed oil, black seed oil, argan oil, pumpkin seed oil, mct oil or any combination thereof, such as olive oil or coconut oil or any combination thereof to form an ozonated oil. The amount of ozonated oil in the embodiments of the present disclosure can be in the range of from about 0.25 wt % to about 95 wt %, 0.5 wt % to 30 wt % (i.e., 5% ozonated olive oil in, for example a solution w coconut oil).

Ozonated oils, for example, olive oil, castor oil, coconut, hemp oil, sesame oil, sunflower oil, canola oil, avocado oil, grapeseed oil, black seed oil, argan oil, pumpkin seed oil, mct oil, almond oil, castor oil, jojoba oil or any combination thereof can be produced using a variety of methods including, for example, ozonation, of different vegetable oils, such as castor oil (U.S. Pat. No. 1,210,949), olive oil (U.S. Pat. No. 2,356,062) and soybean oil (U.S. Pat. No. 3,504,038). Another example includes an ozone generator connected to an aerator that is placed in a glass jar containing oil. The aerator bubbles the ozone from the ozone generator through the oil to make an ozonated oil.

Ozonated oil is known to have an antibacterial effect on the dental surfaces of both gums and teeth. It is also known to help break up biofilm and be effective in controlling harmful bacteria. Studies have shown olive oil that has been ozonized is an effective tool for combating gum disease and restoring gum health. It has also been shown in the prevention of enamel demineralization (caries) by application of ozonized olive oil. An in vivo study investigated the effect of ozonized olive oil gel on enamel the phenomenon of demineralization in people undergoing orthodontic treatment. The reversal of enamel demineralization after orthodontic treatment has been shown to be facilitated by the use of an ozonized olive oil gel.

This use of ozone in an oil (i.e., an ozonated oil) combined with a remineralization and anti-sensitivity agent can help patients when applied chairside in a dentist's office or at home to dental tissues. The goal is to promote or maintain healthy gums, promote healing, promote remineralization of enamel and reduce sensitivity. Another benefit can be brightening of the tooth color.

Embodiments of the present disclosure may include one or more antioxidants or preservatives, such as, for example, tocopherols (vitamin E). The amount of tocopherols can be in the range of from about 0.2 wt % to about 3 wt %.

Embodiments of the present disclosure can also include additional ingredients such as a chemical that reduces bacterial contamination, for example, grapefruit seed extract, magnolia bark extract, citrus extracts or tea tree oil. A plant extract that can be used as a replacement for triclosan, a chemical that reduces bacterial contamination. However, some studies have shown that tea tree oil can cause antibiotic-resistant bacteria to develop. The amount of tea tree oil (or GSE, magnolia bark extract, or citrus extracts) can be in the range of from about 0.05 wt % to about 5 wt %.

Embodiments of the present disclosure can also include additional ingredients such as an ingredient that can help with bad breath and tooth decay, for example, magnolia bark extract, an ingredient that can help with bad breath and tooth decay. It's often found in mouthwashes, chewing gum, and toothpaste. The amount of magnolia bark extract can be in the range of from about 0.05 wt % to about 5 wt %.

Embodiments of the present disclosure can include compositions that are a toothpaste (i.e., dentifrice), varnish coating (a resin is a liquid that can harden with a photo initiator or a chemical initiator that will create a barrier between the tooth structure and the saliva) mouthrinse or serum (a “tooth serum” is a liquid oral care product, typically applied directly to teeth with a brush or applicator, designed to, for example, remineralize tooth enamel, reduce sensitivity, strengthen teeth, and sometimes even help brighten them by filling in microscopic cracks and imperfections). The toothpaste embodiment may include an abrasive. Suitable abrasives can include, for example, sodium bicarbonate granular, calcium carbonate, calcium phosphate, bentonite (a.k.a. bentonite clay), kaolin clay, hydrated silica and mixtures thereof. Abrasives can be generally used at levels of from about 10 wt % to about 60 wt %, by weight of the composition.

Embodiments of the present disclosure include compositions having a pH of between about 5 to about 8.95 or between about 6 to about 7.

A dentifrice embodiment of the present disclosure can include, for example, a toothpaste base such as a mixture of glycerin, calcium carbonate and or silica, cellulose or xanthan gum and or sorbitol, and a surfactant such as sodium methyl coco taurate or sodium lauroyl sarcosinate. Additional ingredients for dentifrice embodiments of the present disclosure can include, for example, xylitol, stevia or monk fruit sweetener. Physical stain removal ingredients such as hydrated silica or calcium carbonate or a mix thereof.

An embodiment of the present disclosure can include, as well as other components included in the present disclosure, water, hydrated silica, glycerin, sorbitol, xylitol, sodium methyl cocoyl taurate, cellulose gum, nano HAP (hydroxyapatite), calcium carbonate, CoQ10, stevia, spearmint flavor, vitamin E tocopherol, ozonated olive oil, calcium glycerophosphate cholecalciferol, zinc chloride and vitamin K2 (menatetrenone).

Embodiments of the present disclosure also include methods of treatment using the embodiment compositions of the present disclosure. Such methods of treatment can include application of the embodiment compositions of the present disclosure with by a dentist in a dentist's office to one or more dental surfaces of a patient or by the patient themselves to one or more tooth surfaces. Embodiments of the present disclosure can include serum, varnish coating, or mouth rinse compositions that are administered by an applicator (e.g., a brush or finger) or dental tray. Serums can also be administered by placing them on the tongue or directly to teeth surfaces. Varnish coating can be applied at dental office chairside by a dentist with a brush to tooth or teeth or at home with brush. After application with a mouth rinse or serum including embodiment of the present disclosure, the patient can, for example, swish the composition in their mouth of the patient to coat one or more outer dental surfaces of the teeth from about 2 to about 20 minutes or about 5 minutes followed by expelling or spitting out the excess of the composition and then not eating, drinking or brushing until then next morning. No rinsing or expelling is needed if the embodiment composition is applied by a brush or finger. If treatment using the embodiment composition is applied to the patient's tooth surfaces by a wearing tray or placed on the tongue or as a mouth rinse, the treatment can be for about 20 minutes, about 10 minutes or overnight after the wearing tray containing the composition is placed in the patient's mouth followed by removing the wearing tray from the patient's mouth (if applicable) and the patient spitting or expelling the excess composition from the patient's mouth where the patient should not eat or drink for about 30 minutes thereafter. Such treatment using a mouth rinse or serum for home use can be applied on a daily basis.

Such methods of treatment can include application of the embodiment compositions of the present disclosure that are a toothpaste where the toothpaste is applied to one or more tooth surfaces using an applicator (e.g., a toothbrush). Such treatment can be administered at least twice a day followed by expelling or spitting out the excess of the composition.

Another embodiment of the disclosure relates to a kit for home use of a composition of an embodiment of the present disclosure.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental novel features of the disclosure as applied to the exemplary embodiments thereof, it will be understood that various omissions and substitutions and changes in the form and details of devices and methods illustrated, and in their operation, may be made by those skilled in the art without departing from the spirit or scope of the disclosure. Moreover, it is expressly intended that all combinations of those elements and/or method steps, which perform substantially the same function in substantially the same way to achieve the same results, are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. It is the intention, therefore, to be limited only as indicated by the scope of the claims appended hereto.

1. A composition comprising:
a. molybdenum; and
b. a remineralization and anti-sensitivity agent.

2. The composition according to claim 1, wherein the remineralization and anti-sensitivity agent is HAP (hydroxyapatite).

3. The composition according to claim 2, wherein the amount HAP (hydroxyapatite) is in the range of from about 0.1 wt % to about 20 wt %.

4. The composition according to claim 1, wherein the composition further includes at least one flavoring agent or at least one sweetening agent.

5. The composition according to claim 1, wherein the composition is in the form of a toothpaste, mouth rinse, varnish coating or a serum.

6. The composition according to claim 2, wherein the HAP (hydroxyapatite) can be a nano size, a micro size. a regular size or a mixture of at least two of the sizes.

7. The composition according to claim 2, wherein the HAP (hydroxyapatite) can be a sphere shape, a rod shape or a combination thereof.

8. The composition according to claim 1, wherein the composition has a pH of from about 5 to about 8.95.

9. The composition according to claim 4, wherein the at least one sweetening agent is saccharin, dextrose, sucrose, sucralose, lactose, maltose, levulose, aspartame, xylitol, stevia, erythritol, sodium cyclamate, D-tryptophan, dihydrochalcones, acesulfame, glycine, monk fruit or mixtures of two or more thereof.

10. A method for strengthening teeth, comprising, applying a composition to one or more tooth surfaces in the mouth of a patient, the composition including:
i. molybdenum; and
ii. a remineralization and anti-sensitivity agent.

11. The method according to claim 10, wherein the composition is a toothpaste and the toothpaste is applied at least 2 times a day.

12. The method according to claim 10, wherein the remineralization and anti-sensitivity agent is HAP (hydroxyapatite).

13. The method according to claim 12, wherein the amount HAP (hydroxyapatite) is in the range of from about 0.1 wt % to about 20 wt %.

14. The method according to claim 10, wherein the composition further includes at least one flavoring agent or at least one sweetening agent.

15. The method according to claim 10, wherein the composition is in the form of a toothpaste, varnish coating, mouthrinse or a serum.

16. The method according to claim 12, wherein the HAP (hydroxyapatite) can be a nano size, a micro size. a regular size or a mixture of at least two of the sizes.

17. The method according to claim 12, wherein the HAP (hydroxyapatite) can be a sphere shape, a rod shape or a combination thereof.

18. The method according to claim 10, wherein the composition has a pH of from about 5 to about 8.95.

19. The method according to claim 14, wherein the at least one sweetening agent is saccharin, dextrose, sucrose, sucralose, lactose, maltose, levulose, aspartame, xylitol, stevia, erythritol, sodium cyclamate, D-tryptophan, dihydrochalcones, acesulfame, glycine, monk fruit or mixtures of two or more thereof.

20. The method according to claim 10, wherein the composition is a serum and the serum is applied for about 10 minutes to about overnight.
